(19) United States
(12) Patent Application Publication
(10) Pub. No.: US 2025/0279200 A1
(43) Pub. Date: Sep. 4, 2025

(54) Method and System for Establishing Death in Hemodialysis Patients Prediction Model, and Method and Non-transitory Computer Readable Medium for Predicting Death in Hemodialysis Patients

(71) Applicant: MAYAMINER COMPANY LTD., Taipei City (TW)

(72) Inventors: CHIEH-LI YEN, Taoyuan City (TW); CHIH-HSIANG CHANG, New Taipei City (TW)

(21) Appl. No.: 18/674,176

(22) Filed: May 24, 2024

(30) Foreign Application Priority Data
Feb. 29, 2024 (TW) 113107246

(51) Int. Cl. G16H 50/20; G16H 50/30

(52) U.S. Cl. CPC G16H 50/20; G16H 50/30

(57) ABSTRACT

The present invention provides a method and a system for establishing death in hemodialysis patients prediction model, and a method and a non-transitory computer readable medium for predicting death in hemodialysis patients, which uses multiple factors to conduct a comprehensive analysis of probability of death of patients so the mortality risk can be more accurately predicted. In response to the prediction, the hemodialysis facilities can act in advance and improve the quality of care for hemodialysis patients.

### FIELD

The present invention relates in general to a method and a system for establishing a prediction model, and more particularly to a method and a system for establishing death in hemodialysis patients prediction model by using big data, and a method and a non-transitory computer readable medium for predicting death in hemodialysis patients.

### BACKGROUND

According to the statistics of the Ministry of Health and Welfare of ROC (Taiwan) in 2021, there are about 88,000 people suffering from end-stage renal disease (ESRD) and receive long-term hemodialysis in Taiwan. Besides, by World Health Organization's estimate, there are about 4 million people suffering from end-stage renal disease and 2.8 million people receiving long-term hemodialysis worldwide, which is a considerable figure.

In addition, patients receiving hemodialysis are often elderly and suffering from multiple chronic diseases at the same time, while receiving long-term hemodialysis may also cause multiple problems such as nutrient loss, sarcopenia, arteriosclerosis and blood pressure fluctuation. According to the Kidney Disease in Taiwan Annual Report (TWRDS), the mortality rate of hemodialysis patients is higher than that of healthy people of the same age, and the average five-year survival rate of hemodialysis patients is only about 53%. In the aspect of average life expectancy, the average life expectancy of male patients is about 71 years, and that of female patients is about 75 years, which is 10 years less than that of the general public. Thus, Taiwan Society of Nephrology (TSN) and nephrologists keep investing in research to reduce the mortality of hemodialysis patients and prolong their lifespan.

In addition, due to arteriosclerosis, deterioration of cardiac function and hypoimmunity, hemodialysis patients often die unpredictability if cardiovascular disease or complicated infection suddenly occurs during hemodialysis, which catches their families off guard. Therefore, if the risk of death within six months or one year in hemodialysis patients can be predicted, clinicians may have an opportunity to check up on the patient's physical condition in advance, and take action in time to prevent the patients from death.

At present, hemodialysis patients should have blood tests every month to examine nutritional indicators such as hemoglobin and albumin and arteriosclerosis indicators such as calcium, phosphorus, and intact-PTH. However, the correlation of these blood biochemical indices and patients' mortality risk are mostly analyzed only with single factors. Furthermore, the interpretation of these blood test results relies on the personal experience of the doctors, which results in the hemodialysis facilities cannot effectively predict the mortality risk of hemodialysis patients due to the lack of standardized interpretation method.

Accordingly, a need exists for a method for accurately predicting death in hemodialysis patients that enables hemodialysis facilities to take action in time to prevent the patients from death.

### SUMMARY

The main purpose of the present invention is to make use of multiple factors to predict probability of death within six months or one year in hemodialysis patients. The hemodialysis facilities can further take the prediction as reference to determine the patients' following treatment or referral.

To achieve the above purpose, the present invention provides a method for establishing a death in hemodialysis patients prediction model, comprising the following steps, by a processor: obtaining a training set and a validation set, wherein the training set and the validation set are corresponding to a plurality of variables; using a univariate Cox proportional hazard regression model to analyze the variables of the training set to identify multiple potential variables, wherein the potential variables have the potential to be used to predict probability of death within six months or one year in hemodialysis patients; using a multivariate Cox proportional hazard regression model to build a prediction model, wherein the prediction model is configured to calculate probability of death within six months or one year in hemodialysis patients corresponding to the potential variables; evaluating discrimination and calibration of prediction model; using the validation set to validate the prediction model; and selecting a plurality of related variables, that make the prediction model with the best discrimination and calibration and validated, to construct and store a death in hemodialysis patients prediction model.

The present invention further provides a system for establishing a death in hemodialysis patients prediction model comprising a processor; and a memory device including instructions that, when executed, cause the processor to perform the method for establishing a death in hemodialysis patients prediction model described above.

The present invention provides another method for predicting death in hemodialysis patients, comprising the following steps, by a processor: obtaining actual measurement data of a hemodialysis patient, wherein the actual measurement data comprising a plurality of actual measurement numerical values of the hemodialysis patient and corresponding to the related variables of the death in hemodialysis patients prediction model established by the method described above; inputting the actual measurement data into the death in hemodialysis patients prediction model to obtain a predicted probability of death in the hemodialysis patient within six months or one year.

The present invention further provides a non-transitory computer readable medium storing one or more computer-executable instructions that, when executed by a processor, cause a computer system to: obtain actual measurement data of a hemodialysis patient, wherein the actual measurement data comprising a plurality of actual measurement numerical values of the hemodialysis patient and corresponding to the related variables of the death in hemodialysis patients prediction model established by the method described above; input the actual measurement data into the death in hemodialysis patients prediction model to obtain a predicted probability of death in the hemodialysis patient within six months or one year.

The followings are beneficial effects of the present invention. 1. By analyzing with multiple factors, the prediction can be more accurately made than that is analyzed with single factor or doctors' personal experience. 2. The prediction method can be performed by only one person and the process of it usually takes less than 10 minutes, which saves manpower and time. 3. The probability of death within six months or one year can be calculated merely based on the general information, routinely collected data and blood biochemical indices values of hemodialysis patients. The hemodialysis facilities can act in advance and improve the quality of care for hemodialysis patients.

### BRIEF DESCRIPTION OF THE DRAWINGS

FIG. 1 is a flow diagram illustrating the method for establishing a death in hemodialysis patients prediction model disclosed herein.

FIG. 2 is a histogram illustrating the comparing of the difference in predicted mortality and real mortality between the patient groups with different mortality risks in the training set.

### DETAILED DESCRIPTION

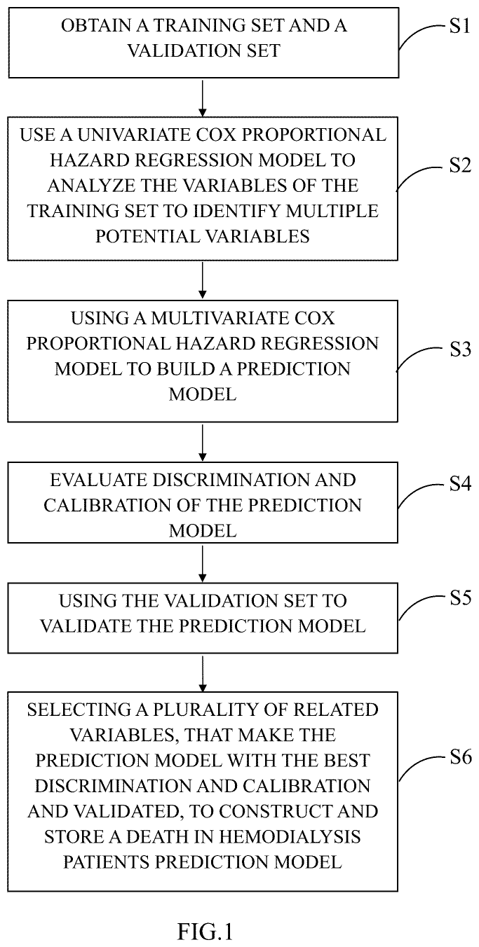

FIG. 1 illustrates a flowchart of the method for establishing a death in hemodialysis patients prediction model, including steps S1-S6.

Depicted as step S1, a processor obtains a training set and a validation set. The training set and the validation set are corresponding to a plurality of variables, and data of the training set and data of the validation set may be separately obtained from hemodialysis patients in different hemodialysis facilities. In addition, the data collected in the training set and the validation set are the data prescribed by the Taiwan Society of Nephrology and meet the requirements of the International Society of Nephrology (ISN) for long-term follow-up of hemodialysis patients, and can be collected by Clinical Biochemistry Analyzer of a general medical institution.

The variables may include: demographic data, primary renal disease data, co-morbidities, hemogram and biochemical profiles, KT/V and urea reduction ratio, time and cause of death in patients. That is, each training data of the training set can include detection values corresponding to the variables listed above, and each validation data of the validation set can include detection values corresponding to the variables listed above.

Depicted as step S2, the processor uses a univariate Cox proportional hazard regression model to analyze the variables of the training set to identify multiple potential variables, wherein the potential variables have the potential to be used to predict probability of death within six months or one year in hemodialysis patients. The variables with a P value less than 0.15 in the univariate Cox proportional hazards regression model may be defined as potential variables.

Depicted as step S3, the processor uses a multivariate Cox proportional hazard regression model to build a prediction model, wherein the prediction model is configured to calculate probability of death within six months or one year in hemodialysis patients corresponding to the potential variables.

Depicted as step S4, the processor evaluates discrimination and calibration of the prediction model. A receiver operating characteristic (ROC) curve may be generated and the area under curve (AUC) may be calculated to evaluate the discrimination of the prediction model. AUC≥0.7 represents acceptable discrimination, and AUC≥0.8 represents excellent discrimination.

The difference in predicted mortality and real mortality between the patient groups with different mortality risks in the training set may be compared to evaluate the calibration of the prediction model. That is, to determine whether the prediction model has stable performance in the patient groups with high, medium, and low mortality risk, respectively.

Depicted as step S5, the processor uses the validation set to validate the prediction model and compares whether the prediction results of the prediction model are consistent with the prediction results of the validation prediction model constructed with the validation set. Specifically, the processor uses the validation set to build a validation prediction model and calculate an AUC value of the validation prediction model. Then, the processor predicts the death of the validation set by using the prediction model constructed by the potential variables and calculates an AUC value. Finally, the processor compares the AUC values between the two. If the AUC values of the two is similar, it means that the prediction capability of the two is comparable, and the prediction model is validated.

Depicted as step S6, the processor selects a plurality of related variables, that make the prediction model with the best discrimination and calibration and validated, to construct and store a death in hemodialysis patients prediction model, wherein the death in hemodialysis patients prediction model is used to calculate probability of death within six months or one year in hemodialysis patients.

By way of example, the de-linked data of patients receiving hemodialysis at hemodialysis facilities for end-stage renal disease from 2010 to 2018 were collected, and the ineligible data such as missing values, younger than 20 years old or older than 90 years old, kidney transplantation done, or not available data were excluded. About 17,359 patients' data were divided into a training set and about 13,656 patients' data were divided into a validation set.

In this working example, the following related variables were selected to build a death in hemodialysis patients prediction model using a multivariate Cox proportional hazard regression model: the patients' age, medical history including: atrial fibrillation, coronary artery disease, peripheral arterial disease, dementia, malignancy, hospitalization due to infection within one year, and routinely blood biochemical indices values including: creatinine, intact-PTH, high-sensitivity c-reactive protein, alkaline phosphatase, high-density lipoprotein cholesterol, glycated hemoglobin, albumin, aspartate transaminase, white blood cells count, platelet count, Kt/V, etc. All the related variables all can be obtained in general hemodialysis facilities.

In terms of discrimination, the AUC value of the death in hemodialysis patients prediction model built with the related variables mentioned above reached 0.808, indicating the prediction model has excellent discrimination.

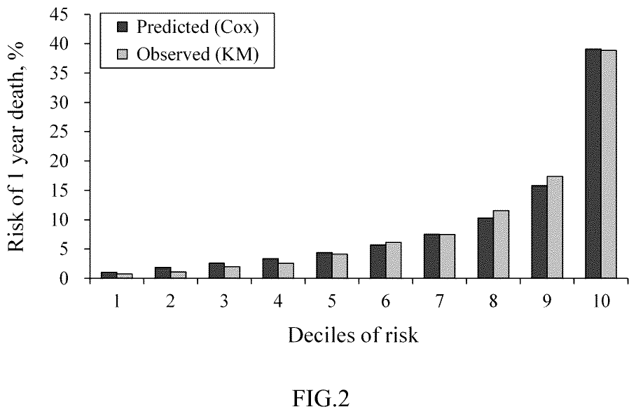

In terms of calibration, the comparing of the difference in predicted mortality and real mortality between the patient groups with different mortality risks in the training set is illustrated with reference to FIG. 2. Number 1 to 10 in horizontal axis represents the deciles of death risk from low to high. The vertical axis of the black histogram (Cox) represents the risk of death within one year (%). The vertical axis of the gray histogram (Observed (KM)) represents the real mortality rate of the patients in their group in the training set. According to FIG. 2, the prediction model has stable calibration from low risk to high risk patients. Moreover, the prediction result of patients with the highest mortality rate is the most accurate. These patients also need the advance warning of death in hemodialysis patients prediction model established by the present invention the most. The calibration of the prediction model in this patients group is the highest as well.

Next, a validation prediction model is built using the validation set, and the AUC value of the validation prediction model is 0.792. Comparatively, the AUC value of the death in hemodialysis patients prediction model built with the related variables mentioned above, when it is used to predict the death of the validation set, reaches 0.789, which is equivalent to the prediction capability of the validation prediction model generated by the validation set. Therefore, the death in hemodialysis patients prediction model is validated, can be popularized to other hemodialysis facilities and maintain robust prediction capability.

According to the method for establishing a death in hemodialysis patients prediction model of the present invention, big data can be used to build a prediction model that effectively predicts the probability of death in hemodialysis patients within six months or one year. On top of that, multiple factors are used to conduct a comprehensive analysis of the mortality risk of patients, which is more accurate than the prediction analyzed with single factor or doctors' personal experience. And the prediction model also has a robust prediction capability in the patients groups with different mortality risks. Its verification also proves that the prediction model is suitable for different hemodialysis facilities.

The present invention further provides a system for establishing a death in hemodialysis patients prediction model comprising a processor; and a memory device including instructions that, when executed, cause the processor to perform the method for establishing a death in hemodialysis patients prediction model described above. The instructions may be software program or machine executable code.

The present invention provides another method for predicting death in hemodialysis patients, comprising the following steps, by a processor: obtaining actual measurement data of a hemodialysis patient, wherein the actual measurement data comprising a plurality of actual measurement numerical values of the hemodialysis patient and corresponding to the related variables of the death in hemodialysis patients prediction model established by the method described above; and inputting the actual measurement data into the death in hemodialysis patients prediction model to obtain a predicted probability of death in the hemodialysis patient within six months or one year.

In a preferred embodiment, the death in hemodialysis patients prediction model is transformed into a Simplified Score System to facilitate the application of the users.

For example, if the death in hemodialysis patients prediction model obtains the actual measurement data of a hemodialysis patients and calculates that the probability of death within one year in the patient is higher than 50%, the hemodialysis facilities can check up on the patient's blood biochemical indices values in advance. If malnutrition occurs, the medical team can consult a dietitian or give the patient nutritional supplements during hemodialysis. If there is severe arteriosclerosis or blood pressure fluctuation, the patient can be transferred to a cardiologist for further examination and treatment as soon as possible. Such early treatment is expected to prolong the life expectancy of hemodialysis patients. On the other hand, if the patient is elderly or has comorbidities such as dementia, malignancy, or multiple-organ failure, and is not expected to survive for a long time, the prediction can also help the family to make preparations beforehand and not to be caught off guard when the patient dies suddenly.

According to the method for predicting death in hemodialysis patients of the present disclosure, only one medical personnel is needed, which may be a nurse, an assistant, etc., to calculate the probability of death within six months or one year in hemodialysis patients after entering the required information. With the computing capability of today's computer device, the prediction process can usually take less than 10 minutes, which saves manpower and time. The prediction results can further be provided to the hemodialysis facilities for reference, so the hemodialysis facilities can act in advance and improve the quality of care for hemodialysis patients.

The present invention further provides a non-transitory computer readable medium storing one or more computer-executable instructions that, when executed by a processor, cause a computer system to: obtain actual measurement data of a hemodialysis patient, wherein the actual measurement data comprising a plurality of actual measurement numerical values of the hemodialysis patient and corresponding to the related variables of the death in hemodialysis patients prediction model established by the method described above; input the actual measurement data into the death in hemodialysis patients prediction model to obtain a predicted probability of death in the hemodialysis patient within six months or one year. The non-transitory computer readable medium may be hard disks, optical discs, USB flash drives, read-only memory (ROM), flash memory cards, network-accessible databases, or other types of computer readable media that can store data that is accessible by a computer.

In summary, the present invention has the following beneficial effects. 1. By analyzing with multiple factors, the prediction can be more accurately made than that is analyzed with single factor or doctors' personal experience. 2. The prediction method can be performed by only one person and the process of it usually takes less than 10 minutes, which saves manpower and time. 3. The probability of death within six months or one year can be calculated merely based on the general information, routinely collected data and blood biochemical indices values of hemodialysis patients. With the prediction, the hemodialysis facilities can act in advance and improve the quality of care for hemodialysis patients.

The description above is merely preferred embodiments of the present invention, and it does not limit the scope of patent protection; therefore, modifications of equivalent effects based on the present specification should be deemed to be included in the scope of the present invention.

1. A method for establishing a death in hemodialysis patients prediction model, comprising the following steps, by a processor:
obtaining a training set and a validation set, wherein the training set and the validation set are corresponding to a plurality of variables;
using a univariate Cox proportional hazard regression model to analyze the variables of the training set to identify multiple potential variables, wherein the potential variables have the potential to be used to predict probability of death within six months or one year in hemodialysis patients;
using a multivariate Cox proportional hazard regression model to build a prediction model, wherein the prediction model is configured to calculate probability of death within six months or one year in hemodialysis patients corresponding to the potential variables;
evaluating discrimination and calibration of the prediction model;
using the validation set to validate the prediction model; and
selecting a plurality of related variables, that make the prediction model with the best discrimination and calibration and validated, to construct and store a death in hemodialysis patients prediction model.

2. The method of claim 1, wherein the step for using a univariate Cox proportional hazard regression model to analyze the variables of the training set further comprises defining the variables with a P value less than 0.15 in the univariate Cox proportional hazards regression model as potential variables.

3. The method of claim 1, wherein the step for evaluating discrimination and calibration of the prediction model further comprises calculating an AUC (area under curve) value to evaluate the discrimination of the prediction model, and comparing the difference in predicted mortality and real mortality between the patient groups with different mortality risks in the training set to evaluate the calibration of the prediction model.

4. The method of claim 1, wherein the step for using the validation set to validate the prediction model further comprises:
using the validation set to build a validation prediction model and calculating an AUC value of the validation prediction model;
predicting the death of the validation set by using the prediction model constructed by the potential variables and calculating an AUC value; and
comparing the AUC values between the two.

5. The method of claim 1, wherein data of the training set and data of the validation set are separately obtained from hemodialysis patients in different hemodialysis facilities.

6. The method of claim 1, wherein the variables of the training set and the validation set including demographic data, primary renal disease, co-morbidities, hemogram and biochemical profiles, KT/V and urea reduction ratio, time and cause of death in patients.

7. A system for establishing a death in hemodialysis patients prediction model, comprising:
a processor;
a memory device including instructions that, when executed, cause the processor to perform operations comprising:
obtaining a training set and a validation set, wherein the training set and the validation set are corresponding to a plurality of variables;
using a univariate Cox proportional hazard regression model to analyze the variables of the training set to identify multiple potential variables, wherein the potential variables have the potential to be used to predict probability of death within six months or one year in hemodialysis patients;
using a multivariate Cox proportional hazard regression model to build a prediction model, wherein the prediction model is configured to calculate probability of death within six months or one year in hemodialysis patients corresponding to the potential variables;
evaluating discrimination and calibration of the prediction model;
using the validation set to validate the prediction model; and
selecting a plurality of related variables, that make the prediction model with the best discrimination and calibration and validated, to construct and store a death in hemodialysis patients prediction model.

8. The system of claim 7, wherein the instructions when executed, cause the processor to further define the variables with a P value less than 0.15 in the univariate Cox proportional hazards regression model as potential variables.

9. The system of claim 7, wherein the instructions when executed, cause the processor to further calculate an AUC value to evaluate the discrimination of the prediction model, and compare the difference in predicted mortality and real mortality between the patient groups with different mortality risks in the training set to evaluate the calibration of the prediction model.

10. The system of claim 7, wherein the instructions when executed, cause the processor to further:
use the validation set to build a validation prediction model and calculate an AUC value of the validation prediction model;
predict the death of the validation set by using the prediction model constructed by the potential variables and calculate an AUC value; and
compare the AUC values between the two.

11. The system of claim 7, wherein data of the training set and data of the validation set are separately obtained from hemodialysis patients in different hemodialysis facilities.

12. The system of claim 7, wherein the variables of the training set and the validation set including demographic data, primary renal disease, co-morbidities, hemogram and biochemical profiles, KT/V and urea reduction ratio, time and cause of death in patient.

13. A method for predicting death in hemodialysis patients, comprising the following steps, by a processor:
obtaining actual measurement data of a hemodialysis patient, wherein the actual measurement data comprising a plurality of actual measurement numerical values of the hemodialysis patient and corresponding to the related variables of the death in hemodialysis patients prediction model established by the method of claim 1; and
inputting the actual measurement data into the death in hemodialysis patients prediction model to obtain a predicted probability of death in the hemodialysis patient within six months or one year.

14. The method of 13, wherein the death in hemodialysis patients prediction model is transformed into a simplified score system.

15. A non-transitory computer readable medium storing one or more computer-executable instructions that, when executed by a processor, cause a computer system to:
obtain actual measurement data of a hemodialysis patient, wherein the actual measurement data comprising a plurality of actual measurement numerical values of the hemodialysis patient and corresponding to the related variables of the death in hemodialysis patients prediction model established by the method of claim 1; and
input the actual measurement data into the death in hemodialysis patients prediction model to obtain a predicted probability of death in the hemodialysis patient within six months or one year.

16. The non-transitory computer readable medium of claim 15, wherein the death in hemodialysis patients prediction model is transformed into a simplified score system.
